(19) United States
(12) Patent Application Publication
(10) Pub. No.: US 2023/0338510 A1
(43) Pub. Date: Oct. 26, 2023

(54) NOVEL CORONAVIRUS TANDEM EPITOPE POLYPEPTIDE VACCINE AND USE THEREOF

(71) Applicant: SHANGHAI INSTITUTE OF MATERIA MEDICA, CHINESE ACADEMY OF SCIENCES, Shanghai (CN)

(72) Inventors: Likun GONG, Shanghai (CN); Jin REN, Shanghai (CN); Qiuping QIN, Shanghai (CN); Weiliang ZHU, Shanghai (CN); Yiru LONG, Shanghai (CN); Zhijian XU, Shanghai (CN); Jianhua SUN, Shanghai (CN); Tingting LIU, Shanghai (CN); Guangyi JIN, Shanghai (CN); Jianping ZUO, Shanghai (CN); Wei HUANG, Shanghai (CN)

(21) Appl. No.: 18/010,522

(22) PCT Filed: Jun. 11, 2021
(86) PCT No.: PCT/CN2021/099860

§ 371 Date: Dec. 15, 2022

(30) Foreign Application Priority Data
Jun. 15, 2020 (CN) 202010546749.1

(51) Int. Cl. A61P 31/14; A61K 39/215; A61K 39/00

(52) U.S. Cl. CPC A61K 39/215; A61K 39/4611; A61K 39/4613; A61K 39/4614; A61K 39/4615; A61K 39/464838; A61P 31/14; A61K 2039/575; A61K 2039/70

(57) ABSTRACT

Provided are a tandem epitope polypeptide vaccine for novel coronavirus and use thereof. Specifically, a vaccine polypeptide for novel coronavirus pneumonia is provided on the basis of analysis and study of the RBD sequence and structural information of the S protein of SARS-CoV-2. Said vaccine polypeptide comprises the following elements connected in series: a generic Th epitope sequence, a B cell epitope sequence and a T cell epitope sequence. The B cell epitope and the T cell epitope have an amino acid sequence from the RBM region of the S protein of SARS-CoV-2. Provided are a vaccine composition containing said vaccine polypeptide and use thereof. Experiments show that the vaccine polypeptide of the present invention can enable cynomolgus monkeys to initiate strong cellular and humoral immunity, and to generate neutralizing antibodies that block the binding of RBD and ACE2, and can be used for preventing and treating novel coronavirus pneumonia.

### TECHNICAL FIELD

The present invention relates to the field of polypeptide drug and polypeptide vaccine, in particular, to a novel coronavirus tandem epitope polypeptide vaccine and use thereof.

### BACKGROUND

Novel coronavirus pneumonia (Corona virus disease 2019, COVID-19) caused by coronavirus SARS-CoV-2 is extremely contagious. However, there is still a lack of clearly effective drugs and measures for prevention and treatment of COVID-19, and supportive treatment and symptomatic treatment are the main focus clinically.

Establishing herd immunity to SARS-CoV-2 through vaccines is the ultimate way to control and block COVID-19 epidemic. At present, many types of COVID-19 vaccines have been in preclinical and clinical trials, including live attenuated live vaccines, inactivated virus vaccines, recombinant virus vector vaccines, recombinant protein vaccines, DNA vaccines, RNA vaccines and peptide vaccines, etc..

However, the protective effect of vaccines developed at present is limited, for in presence of problems such as low immunogenicity, safety risk, low population response rate, and difficulty in overcoming virus immune escape and so on.

Therefore, there is an urgent need in the art to develop new vaccines that have a high population response rate and can efficiently stimulate the human body to produce an immune response against SARS-CoV-2, in order to produce blocking anti-SARS-CoV-2 antibodies in vaccinators, thereby providing powerful immune protection.

### SUMMARY OF THE INVENTION

The purpose of the present invention is to provide a new vaccine that have a high population response rate and can efficiently stimulate the human body to produce an immune response against SARS-CoV-2, and then produce blocking anti-SARS-CoV-2 antibodies in vaccinators, thereby providing powerful immune protection.

In the first aspect of the present invention, it provides a vaccine polypeptide of novel coronavirus, which comprises the following elements in tandem: a universal Th epitope sequence, a B-cell epitope sequence and a T-cell epitope sequence, wherein the B-cell epitope and the T-cell epitope have amino acid sequences derived from the RBM region of the SARS-CoV-2 S protein.

In another preferred embodiment, the universal Th epitope sequence comprises a PADRE sequence.

In another preferred embodiment, the PADRE sequence comprises AKFVAAWTLKAAA (positions 1-13 in SEQ ID NO: 1).

In another preferred embodiment, the vaccine polypeptide can stimulate primates and rodents to produce neutralizing antibodies that block the binding of RBD to ACE2.

In another preferred embodiment, the vaccine polypeptide can stimulate primates to produce cellular immunity and humoral immunity.

In another preferred embodiment, the primates include humans and non-human primates.

In another preferred embodiment, the length of the antigenic polypeptide is 40-100 amino acids, preferably 45-80 amino acids.

In another preferred embodiment, the vaccine polypeptide has a structure of Formula I or an oligomer comprising a structure of Formula I:

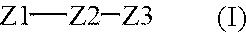

wherein,
Z1, Z2, and Z3 are each independently a universal Th epitope sequence, a B-cell epitope sequence, a T-cell epitope, or a combination thereof;
“-” is a chemical bond or a linker;
and at least one of Z1, Z2, and Z3 is the universal Th epitope sequence; at least one is the B-cell epitope sequence; and at least one is the T-cell epitope.

In another preferred embodiment, Z2 is the B-cell epitope and Z3 is the T-cell epitope.

In another preferred embodiment, Z2 is the T-cell epitope and Z3 is the B-cell epitope.

In another preferred embodiment, the universal Th epitope sequence includes a PADRE sequence.

In another preferred embodiment, the “-” is a peptide bond or a linking peptide.

In another preferred embodiment, the linking peptide is a polyglycine formed by 3-6 glycines.

In another preferred embodiment, the linking peptide is flexible.

In another preferred embodiment, the linking peptide is GGGG (i.e., G₄).

In another preferred embodiment, the lengths of the B-cell epitope sequence and the T-cell epitope are each independently 10-20 amino acids, preferably 12-18 amino acids.

In another preferred embodiment, except for the PARDE sequence and the linking peptide, other sequences in the antigenic polypeptide are derived from the amino acid sequence of the RBM region of the S protein.

In another preferred embodiment, the B-cell epitope and/or T-cell epitope in the antigenic polypeptide has an amino acid sequence derived from the RBD region of the novel coronavirus S protein.

In another preferred embodiment, Z2 and/or Z3 in the antigenic polypeptide has an amino acid sequence derived from the RBM region of the RBD region.

In another preferred embodiment, the RBM region refers to amino acids positions 438-506 of the novel coronavirus RBD protein.

In another preferred embodiment, the antigenic polypeptide “having an amino acid sequence derived from the RBM region of the RBD protein” means that the amino acid sequence of the antigenic polypeptide has a homology (or identity) with the RBM region, and the homology is ≥80%, preferably ≥85%, more preferably ≥90%, and most preferably ≥95%.

In another preferred embodiment, the antigenic polypeptide competitively binds to the human ACE2 protein with the S protein of the novel coronavirus.

In another preferred embodiment, the “competitive binding” means that the antigenic polypeptide (or B-cell epitope and/or T-cell epitope therein) is involved in the binding of the S protein of the novel coronavirus to the human ACE2 protein.

In another preferred embodiment, the competitive binding includes blocking or non-blocking competitive binding.

In another preferred embodiment, the antigenic polypeptide is an artificially synthesized or recombinant antigenic polypeptide.

In another preferred embodiment, in Formula I, Z1 is AKFVAAWTLKAAA (positions 1-13 in SEQ ID NO: 1); Z2 is YGFQPTNGVGYQP (positions 18-30 in SEQ ID NO: 1); Z3 is NYLYRLFRKSNLKPF (positions 18-30 in SEQ ID NO: 1).

In another preferred embodiment, in Formula I, Z1 is AKFVAAWTLKAAA (positions 1-13 in SEQ ID NO: 1); Z2 is NYLYRLFRKSNLKPF (positions 18-30 in SEQ ID NO: 1); Z3 is YGFQPTNGVGYQP (positions 18-30 in SEQ ID NO: 1).

In another preferred embodiment, the antigenic polypeptide is selected from the group consisting of:
(a) a polypeptide having the amino acid sequence shown in SEQ ID NO: 1;
(b) a derivative polypeptide formed by one or more amino acids addition, one or more amino acids substitution, or 1-3 amino acids deletion to the linking peptide of the amino acid sequence of the polypeptide in (a), and the derivative polypeptide has the same or substantially the same function as the polypeptide shown in SEQ ID NO: 1 before derivatization.
(c) a derivative polypeptide formed by one or more amino acids addition, one or more amino acids substitution, or 1-3 amino acids deletion to Z1, Z2 and/or Z3 of the amino acid sequence of the polypeptide in (a), and the derivative polypeptide has the same or substantially the same function as the polypeptide shown in SEQ ID NO: 1 before derivatization.

In another preferred embodiment, the “substantially the same function” means that the derivative polypeptide has substantially the same immunogenicity to stimulate an immune response and that the produced antibodies (including antiserum) have the activity to block the binding of the S protein of the novel coronavirus to the human ACE2 protein.

In another preferred embodiment, the vaccine polypeptide has the amino acid sequence as shown in SEQ ID NO: 1.

In another preferred embodiment, the structure of the antigenic polypeptide is as shown in Formula II:

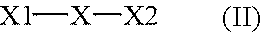

wherein,
(a) X is a core fragment (i.e. oligomer with the structure of Formula I or containing the structure of Formula I); preferably, the sequence of the core fragment is as shown in SEQ ID NO: 1;
(b) X1 and X2 are each independently none, 1, 2, or 3 amino acids, and the total number of amino acids of X1 and X2 is ≤ 4, preferably 3, 2, 1, and more preferably 0 or 1;
(c) “-” represents a peptide bond, peptide linker, or other linker (that is, X1 and X and/or X and X2 are connected by peptide bonds, peptide linkers (such as a flexible linker consisting of 1-15 amino acids) or other linkers).

In another preferred embodiment, X1, X2 are each independently none, K, C, G, L, A.

In another preferred embodiment, X1 is none, K, or C.

In another preferred embodiment, X2 is none, K, or C.

In another preferred embodiment, the antigenic polypeptide has at least one T cell epitope and at least one B cell epitope of the RBD region of the novel coronavirus S protein.

In another preferred embodiment, the antigenic polypeptide has at least one T cell epitope and/or at least one B cell epitope of the RBM region of the novel coronavirus S protein.

In another preferred embodiment, the antigenic polypeptide has at least one T cell epitope, preferably 1, 2, 3 or 4 T cell epitopes, more preferably 1 or 2 T cell epitopes.

In another preferred embodiment, the antigenic polypeptide has at least one B cell epitope, preferably 1, 2, 3 or 4 B cell epitopes, more preferably 1 or 2 B cell epitopes.

In another preferred embodiment, the antigenic polypeptide has 1-2 T cell epitopes and 1-2 B cell epitopes, preferably one T cell epitope and one B cell epitope.

In another preferred embodiment, the T cell epitope includes a CD4+ T cell epitope and a CD8+ T cell epitope.

In another preferred embodiment, the CD4+ T cell epitope primarily activates helper T cells to activate B cells to produce antibodies; the CTL epitope (or CD8+ T cell epitope) activates killer CD8+ T cells to exert antiviral effects.

In another preferred embodiment, the B cell epitopes include linear and conformational B cell epitopes.

In the second aspect of the present invention, it provides an isolated peptide set, comprising at least two vaccine polypeptides of novel coronavirus according to the first aspect of the present invention.

In another preferred embodiment, the peptide set contains at least 2-20 kinds (such as 2, 3, 4, 5, 6, 7, 8, 9, 10, 11, 12 kinds) of the vaccine polypeptide.

In the third aspect of the present invention, it provides a pharmaceutical composition, comprising the vaccine polypeptide of novel coronavirus according to the first aspect of the present invention or the peptide set according to the second aspect of the present invention and a pharmaceutically acceptable carrier.

In another preferred embodiment, the pharmaceutical composition is a vaccine composition.

In another preferred embodiment, the vaccine composition is monovalent or multivalent.

In another preferred embodiment, the pharmaceutical composition further comprises an adjuvant, and various aluminum adjuvants are preferred. The molar or weight ratio of active peptide and adjuvant (such as aluminum) in the composition is between 1:100, preferably between 1:40 and 1:60.

In another preferred embodiment, the pharmaceutical composition includes single drug, compound drugs, or synergistic drugs.

In another preferred embodiment, the dosage form of the pharmaceutical composition is liquid, solid, or gel.

In another preferred embodiment, the pharmaceutical composition is administered in a manner selected from the group consisting of: subcutaneous injection, intradermal injection, intramuscular injection, intravenous injection, intraperitoneal injection, microneedle injection, oral administration, oral and nasal spray or atomization inhalation.

In the fourth aspect of the present invention, it provides a use of the vaccine polypeptide of novel coronavirus according to the first aspect or the peptide set according to the second aspect or the pharmaceutical composition according to the third aspect of the present invention, in the manufacture of a medicament for the prevention of coronavirus SARS-CoV-2 infection or related disease thereof.

In another preferred embodiment, the coronavirus SARS-CoV-2 related disease is selected from the group consisting of: respiratory infection, pneumonia and its complications, and a combination thereof.

In another preferred embodiment, the coronavirus SARS-CoV-2 related disease is novel coronavirus pneumonia (COVID-19).

In the fifth aspect of the present invention, it provides a cell preparation, comprising (a) immune cells immuno-activiated by the vaccine polypeptide of novel coronavirus according to the first aspect of the present invention or the peptide set according to the second aspect of the present invention; and (b) a pharmaceutically acceptable carrier.

In another preferred embodiment, the immune cells are selected from the group consisting of: dendritic cells, natural killer cells (NK), lymphocytes, monocytes/macrophages, granulocytes, and a combination thereof.

In another preferred embodiment, the activation is in vitro activation.

In another preferred embodiment, the in vitro activation includes: culturing the immune cells for a period of time (such as 6-48 hours) in the presence of the vaccine polypeptide to obtain the immuno-activiated immune cells.

In another preferred embodiment, the cell preparation is a liquid preparation comprising living cells.

In another preferred embodiment, the cell preparation is re-infused through intravenous administration.

In the sixth aspect of the present invention, it provides a method for generating an immune response against the coronavirus SARS-CoV-2, which comprises the steps of: administering the vaccine polypeptide of novel coronavirus according to the first aspect, the peptide set according to the second aspect or the pharmaceutical composition according to the third aspect of the present invention to a subject in need thereof.

In another preferred embodiment, the subject includes human or non-human mammals.

In another preferred embodiment, the non-human mammals includes non-human primates (such as monkeys).

In another preferred embodiment, the method induces the production of neutralizing antibodies against the coronavirus SARS-CoV-2 in the subject.

In another preferred embodiment, the neutralizing antibody blocks the binding of coronavirus SARS-CoV-2 to human ACE2 protein.

In the seventh aspect of the present invention, it provides a fusion protein, comprising a carrier protein and the vaccine polypeptide according to the first aspect of the present invention fused to the fusion protein.

In another preferred embodiment, the fusion protein has a structure of Formula IIIa or IIIb:

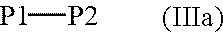

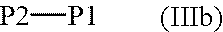

wherein, P1 is the vaccine polypeptide according to the first aspect of the present invention, and P2 is the carrier protein.

In another preferred embodiment, the P1 may be a single vaccine polypeptide, or multiple identical or different vaccine polypeptides (or antigenic polypeptides) in tandem.

In the eighth aspect of the present invention, it provides a pharmaceutical composition, which comprises (a) the fusion protein according to the seventh aspect of the present invention or immune cells immuno-activated by the fusion protein; and (b) a pharmaceutically acceptable carrier.

In the ninth aspect of the present invention, it provides a use of the fusion protein according to the seventh aspect of the present invention or the pharmaceutical composition according to the eighth aspect of the present invention, in the manufacture of a medicament for the prevention of coronavirus SARS-CoV-2 infection or related disease thereof.

It should be understood that within the scope of the present invention, each technical features of the present invention described above and in the following (as examples) may be combined with each other to form a new or preferred technical solution, which is not listed here due to space limitations.

### DESCRIPTION OF DRAWINGS

FIG. 1 shows the interaction structure and key action sites of SARS-CoV-2 S protein RBD region and human ACE2.

FIG. 2 shows the main CD4+T antigenic epitope regions of S protein predicted by NetMHC II software.

FIG. 3 shows the main CD8+T antigenic epitope regions of S protein predicted by NetCTL software.

FIG. 4 shows the linear B cell epitope regions contained in S protein RBD predicted by BepiPred software.

FIG. 5 shows the conformational B cell epitopes contained in S protein RBD predicted by Discotope software.

FIG. 6 shows the epitope tandem structure composition pattern of LP2.

FIG. 7 shows the relative spatial position of the B cell epitopes and T cell epitopes contained in LP2 in the S protein RBD region.

FIG. 8 shows the production of high titers anti-RBD antibodies by cynomolgus monkeys after LP2 immunization.

FIG. 9 shows that the anti-RBD antibodies produced by LP2 immunized cynomolgus monkeys can block the binding of RBD to ACE2.

FIG. 10 shows that LP2 can bind well to human ACE2 protein in vitro.

### DETAILED DESCRIPTION

Upon extensive and intensive studies, based on the analysis of the sequence and structure of the SARS-CoV-2 S protein RBD, the inventors have analyzed the T/B cell epitopes, key sites of interaction, surface features and peptide physical and chemical properties of the S protein, screened and identified vaccine polypeptides that can effectively induce the mammalian organism to produce immune response against the coronavirus SARS-CoV-2 for the first time. Experiments show that the vaccine polypeptide of the present invention can effectively trigger cellular and humoral immunity against SARS-CoV-2 in primates (such as cynomolgus monkeys), thereby producing higher titer neutralizing antibodies that block the binding of RBD to ACE2, so the present invention has potential application prospects in the prevention or treatment of novel coronavirus pneumonia. The present invention has been completed on this basis.

Specifically, based on the analysis of the RBD sequence and structural information of the SARS-CoV-2 S protein, the inventor screened and determined the specific B cell epitope sequences and T cell epitope sequences of the RBD region by determining the CD4+ T/CD8+ T cell epitopes and the linear/conformational B cell epitopes of the S protein, and comprehensively considering the structural surface characteristics, the key sites of interaction with ACE2, and the polypeptide physical and chemical properties of the S protein, etc.. And these epitopes are connected with universal Th epitopes (such as PADRE sequence) in series to form novel tandem epitope polypeptides, which improves the problems with common polypeptides of low immunogenicity and low response rate of people affected by MHC restriction, and takes into account the advantages of high specificity, high safety and easy synthetic production of common polypeptides. Experiments show that the tandem epitope polypeptide of the present invention can enable cynomolgus monkeys to initiate strong cellular and humoral immunity, and produce higher titer neutralizing antibodies that block the binding of RBD and ACE2. At the same time, the tandem epitope polypeptide of the present invention has an optimized structure, and the T/B cell epitope sequences contained therein are located on the action interface between RBD and human ACE2, and unexpectedly can still bind well with human ACE2 after tandem, showing the potential to directly block the interaction between RBD and ACE2.

### Term

### Coronavirus SARS-CoV-2

Coronavirus (CoV) belongs to the nidovirales coronaviridae, which is an enveloped positive-strand RNA virus, and its subfamily includes four genera α, β, δ and γ.

Among the currently known coronaviruses that infect humans, HCoV-229E and HCoV-NL63 belong to the α genus coronavirus, and HCoV-OC43, SARS-CoV, HCoV-HKU1, MERS-CoV and SARS-CoV-2 are all β genus coronaviruses.

The novel coronavirus (SARS-CoV-2) that broke out at the end of 2019 has about 80% similarity with SARS-CoV and 40% similarity with MERS-CoV. It also belongs to the β genus coronavirus.

The genome of this type of virus is a single-stranded positive-stranded RNA, which is one of the RNA viruses with largest genome, encoding replicase, spike protein, capsule protein, envelope protein, and nucleocapsid protein, etc.. In the initial stage of virus replication, the genome is translated into two peptide chains of several thousand amino acids, that is the precursor polyprotein, and then the precursor proteins are cleaved by proteases to produce non-structural proteins (such as RNA polymerase and helicase) and structural proteins (such as spike proteins) and accessory proteins.

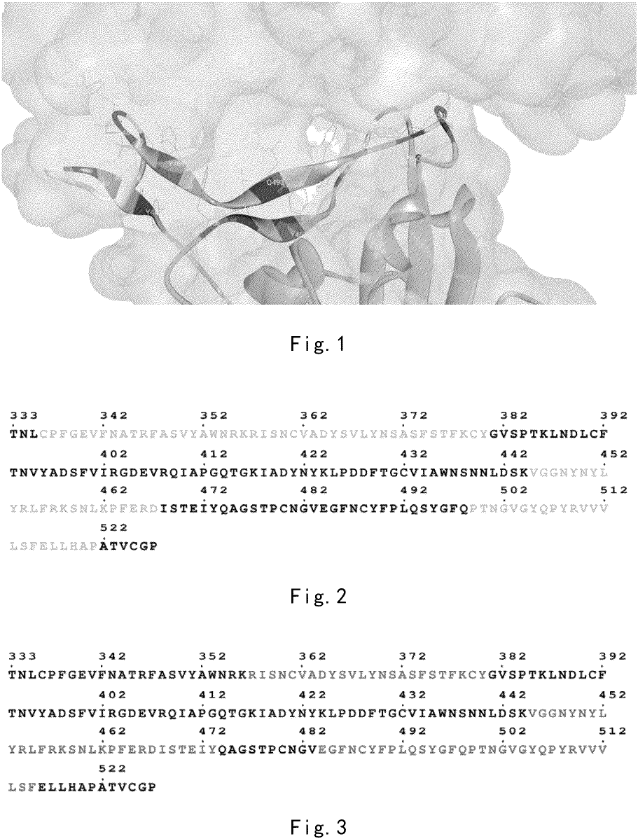

The S protein is a major structural protein of the coronavirus SARS-CoV-2. The schematic diagram of its structure is shown in FIG. 1, wherein, RBD is responsible for binding to human ACE2 receptor, and the RBM region comprises a motif that binds to human ACE2. The amino acid sequence of a typical S protein is shown in SEQ ID No: 2.

MFVFLVLLPLVSSQCVNLTTRTQLPPAYTNSFTRGVYYPDKVFRSSV
LHSTQDLFLPFFSNVTWFHAIHVSGTNGTKRFDNPVLPFNDGVYFASTEK
SNIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRS
YLTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYA
DSFVIRGDEVRQIAPGQTGKIADYNYKLPDDFTGCVIAWNSNNLDSKVGG
NYNYLYRLFRKSNLKPFERDISTEIYQAGSTPCNGVEGFNCYFPLQSYGF QPTNGVGYQPYRVVVLSFELLHAPATVCGPKKSTNLVKNKCVNFNFNGLT
GTGVLTESNKKFLPFQQFGRDIADTTDAVRDPQTLEILDITPCSFGGVSV
ITPGTNTSNQVAVLYQDVNCTEVPVAIHADQLTPTWRVYSTGSNVFQTRA
GCLIGAEHVNNSYECDIPIGAGICASYQTQTNSPRRARSVASQSIIAYTM
SLGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAAR
DLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGK
LQDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDS
FKEELDKYFKNHTSPDVDLGDISGINASVVNIQKEIDRLNEVAKNLNESL
IDLQELGKYEQYIKWPWYIWLGFIAGLIAIVMVTIMLCCMTSCCSCLKGC
CSCGSCCKFDEDDSEPVLKGVKLHYT (SEQ ID No: 2)

The RBD region of the coronavirus SARS-CoV-2 is located at positions 333-527 of the S protein, and a representative amino acid sequence is shown in SEQ ID No: 2 positions 333-527.

>RBD527)

TNLCPFGEVFNATRFASVYAWNRKRISNCVADYSVLYNSASFSTFKC
YGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTP
CNGVEGFNCYFPLQSYGFQPTNGVGYQPYRVVVLSFELLHAPATVCGP (
SEQ ID No: 2 positions333-527)

The RBM region of the coronavirus SARS-CoV-2 is located at positions 438-506 of the S protein, and a representative amino acid sequence is shown in SEQ ID No: 2 positions 438-506.

>RBM506)

SNNLDSKVGGNYNYLYRLFRKSNLKPFERDISTEIYQAGSTPCNGVE
GFNCYFPLQSYGFQPTNGVGYQ (SEQ ID No: 2 positions 438
-506)

It should be understood that in the present invention, the S protein, RBD region and RBM region all include wild type and mutant type.

### Vaccine Polypeptide

In the present invention, “epitope peptide of the present invention”, “vaccine polypeptide of the present invention”, “polypeptide of the present invention”, “tandem epitope polypeptide of the present invention” can be used interchangeably, and refer to the vaccine polypeptide according to the first aspect of the present invention, in particular the polypeptide having a structure of Formula I. It should be understood that the terms include not only one kind of vaccine polypeptide of the present invention, but also peptide set (or peptide combinations) formed by multiple kinds of vaccine polypeptide of the present invention.

Preferably, the vaccine polypeptide of the present invention comprises at least one T cell epitope and at least one B cell epitope. Preferably, in the vaccine polypeptide of the present invention, the CD4⁺T cell epitope mainly activates helper T cells to activate B cells to produce antibodies, and the CTL epitope can activate killer CD8⁺T cells to exert antiviral effects. Linear and conformational B cell epitopes can act on BCR to directly activate B cells to produce antibodies.

In the present invention, vaccine polypeptides also include other forms, such as pharmaceutically acceptable salts, conjugates, or fusion proteins.

In addition, in the present invention, the preferred vaccine polypeptide has the structure shown in Formula II:

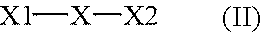

(a) X is a core fragment, wherein the sequence of the core fragment is shown in SEQ ID No: 1;
(b) X1 and X2 are each independently none, 1, 2, or 3 amino acids, and the total number of amino acids of X1 and X2 is ≤ 4, preferably 3, 2, 1, and more preferably 0 or 1;
wherein, X1 and X, X and X2, are connected by peptide bonds, peptide linkers (such as a flexible linker composed of 1-15 amino acids) or other linkers.

In the present invention, the core fragment or vaccine polypeptide includes a derivative polypeptide formed by one or more (e.g., 1-5, preferably 1-3) amino acids addition, one or more (e.g., 1-5, preferably 1-3) amino acids substitution, and/or 1-3 amino acids deletion to any one of sequence shown in SEQ ID No: 1, and the derivative polypeptide has substantially the same function as the original polypeptide before derivatization.

Preferably, the core fragment or vaccine polypeptide includes 1-3 amino acids addition (preferably the N-terminal or C-terminal addition), and/or 1-2 amino acids substitution (preferably conservative amino acid substitution) to SEQ ID No: 1, and still has substantially the same function as the original polypeptide before derivatization.

Preferably, the conservative amino acid substitution is performed according to Table A.

TABLE A

| | | |
|---|---|---|
| Initial residue | Representative substitutions | Preferred substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

As used herein, the peptide set described by the term “peptide set” consists of at least two kinds of vaccine polypeptides of the present invention or derivative polypeptides thereof.

Preferably, the peptide set of the present invention comprises at least 2, 3, 4, 5, 6, 7, 8, 9, or 10 kinds of vaccine polypeptides or derivative polypeptides thereof (including coupled peptides) selected from the first aspect of the present invention; more preferably, the peptide set comprises at least one vaccine polypeptide of SEQ ID NO.: 1 or derivative polypeptides thereof. In addition, the peptide set may also comprise other antigenic peptides or proteins of coronavirus SARS-CoV-2 except SEQ ID NO.: 1.

As used herein, “isolated” refers to the separation of a substance from its original environment (if it is a natural substance, the original environment is the natural environment). For example, the polypeptide in living cells in the natural state is not separated and purified, but the same polypeptide is separated and purified if it is separated from other substances that exist in the natural state.

As used herein, “isolated peptide” means that the polypeptide of the present invention is substantially free of other proteins, lipids, carbohydrates or other substances naturally associated therewith. Those skilled in the art can use standard protein purification techniques to purify the polypeptide of the present invention. The substantially purified polypeptide (fusion protein) can produce a single main band on a non-reducing polyacrylamide gel.

The polypeptide of the present invention may be a recombinant polypeptide or a synthetic polypeptide, preferably a synthetic polypeptide.

In the present invention, when the sequence of the vaccine polypeptide is short (such as ≤ 70aa, more preferably ≤ 60aa), the relevant peptide sequence may be directly synthesized by chemical methods.

When the sequence of the vaccine polypeptide is long or the vaccine polypeptide is provided in the form of a fusion protein, the recombinant method may also be used to obtain the related peptide sequence in large quantities. This usually involves cloning the coding sequence of the antigenic polypeptide or its fusion protein into a vector, and then transferring it into cells, and then isolating the relevant antigenic polypeptide or fusion protein from the proliferated host cell by conventional methods.

### Universal Th Epitope

In the vaccine polypeptide of the present invention, one or more universal Th epitopes may be contained.

In the present invention, by introducing the universal Th epitope, it is unexpectedly found that it can help to improve the population response rate and immunogenicity, and retain the safety, epitope specificity and other characteristics.

Preferably, the universal Th epitope sequence in the present invention is a universal Th epitope sequence that functions in a human body and has a high response rate.

The experimental screening results show that a preferred universal Th epitope sequence includes PADRE sequence.

More preferably, the PADRE sequence comprises AKFVAAWTLKAAA (positions 1-13 in SEQ ID NO: 1).

### B Cell Epitope Sequence and T Cell Epitope

In the vaccine polypeptide of the present invention, at least one B cell epitope sequence and/or at least one T cell epitope may be contained.

In the present invention, the lengths of the B cell epitope sequence and the T cell epitope are not particularly limited, and each may be independently 10-20 amino acids, preferably 12-18 amino acids.

In another preferred embodiment, except for the PARDE sequence and the linking peptide, other sequences in the antigenic polypeptide are derived from the amino acid sequence of the RBM region of the S protein.

In another preferred embodiment, the B cell epitope and/or the T cell epitope in the antigenic polypeptide has an amino acid sequence derived from the RBD region of the novel coronavirus S protein, i.e., Z2 and/or Z3 in the antigenic polypeptide has an amino acid sequence derived from the RBM region of the RBD region.

### Pharmaceutical Composition and Mode of Administration

The present invention also provides a pharmaceutical composition. The pharmaceutical composition of the present invention may be therapeutic or prophylactic (e.g., vaccine). The pharmaceutical composition of the present invention includes an effective amount of the vaccine polypeptide or peptide set of the present invention, or immune cells activated with the vaccine polypeptide (e.g., dendritic cells sensitized with the vaccine polypeptide of the present invention or T cells induced by the dendritic cells), and at least one pharmaceutically acceptable carrier, diluent or excipient.

In another preferred embodiment, the related diseases caused by novel coronavirus SARS-CoV-2 are selected from group consisting of: respiratory tract infection, pneumonia and its complications, and combinations thereof.

In the present invention, these (vaccine) compositions comprise immune antigens (including the vaccine polypeptides, peptide set or derivatives thereof of the present invention), and are usually combined with “pharmaceutically acceptable carriers”, including any carrier that itself does not induce the production of antibodies that are harmful to the individual receiving the composition. Examples of suitable carriers include (but are not limited to) proteins, lipid aggregates (such as oil droplets or liposomes) and so on. These carriers are well known to those of ordinary skill in the art. In addition, these carriers may function as immunostimulants (“adjuvants”).

In addition, the (vaccine) composition of the present invention may also comprise additional adjuvants. Representative vaccine adjuvants include (but are not limited to) the following types: inorganic adjuvants, such as aluminum hydroxide, alum, etc.; synthetic adjuvants, such as synthetic double-stranded polynucleotides (double-stranded polyadenosine acid, uridine acid), levamisole, isopinosine, etc.; oil agents, such as Freund’s adjuvant, peanut oil emulsification adjuvant, mineral oil, vegetable oil, etc..

Generally, the vaccine composition or immunogenic composition may be made into an injectable agent, for example, a liquid solution or suspension; it may also be made into a solid form suitable for being formulated into a solution or suspension or a liquid excipient before injection. The preparation may also be emulsified or encapsulated in liposomes to enhance the adjuvant effect.

The composition may be made into a unit or multiple dosage form. Each dosage form comprises a predetermined amount of active substance calculated to produce the desired therapeutic effect, and suitable pharmaceutical excipients.

The formulated pharmaceutical composition may be administered by conventional routes, including (but not limited to): intravenous, intramuscular, intraperitoneal, subcutaneous, intradermal, oral, or topical administration.

When using a (vaccine) composition, a safe and effective amount of the vaccine polypeptide or peptide set of the present invention is administered to a human, wherein the safe and effective amount is usually at least about 1 ug peptide/kg body weight, and in most cases not more than about 8 mg peptide/kg body weight, preferably the dose is about 1 ug-1 mg peptide/kg body weight. Of course, the specific dosage should also consider factors such as the administration route and the patient’s health status, which are all within the skill range of a skilled physician.

The main advantages of the present invention include:
(a) The vaccine polypeptides used in the present invention can produce neutralizing antibodies against S protein RBD (receptor binding domain) of SARS-CoV-2 in the body of mammals such as primates, and the neutralizing antibodies can block the binding of RBD to ACE2.
(b) The tandem epitope polypeptide of the present invention has an optimized structure. Unexpectedly, the tandem epitope polypeptide of the present invention has a higher binding ability with human ACE2, and has the potential to directly block the binding of the S protein of the SARS-CoV-2 virus to ACE2.

The present invention is further explained below in conjunction with specific example. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

### Example 1 T/B Cell Epitope Screening and Tandem Epitope Polypeptide Design Based on Sequence and Structure Analysis of S Protein RBD

By analyzing the sequence and structure of SARS-CoV-2 S protein RBD, the inventor predicted and analyzed the CD4⁺T/CD8⁺T cell epitopes, linear/conformational B cell epitopes, structural characteristics and interaction key sites of S protein through a variety of computer-aided vaccine design software tools. Based on the analysis results, the appropriate T/B cell epitopes were screened and determined, and these epitopes were connected in tandem with the universal Th epitope PADRE sequence, wherein, the epitopes were connected by four glycines to ensure that they do not interfere with each other.

Specifically, the inventors used the RBD region in the SARS-CoV-2 virus S protein that interacts with human ACE2 as an analysis object, and determined the key sites in the RBD that interacts with ACE2, as shown in FIG. 1.

The inventors also used Allele Frequency Net Database for statistical analysis to obtain the main HLA class II molecular allele types in the world population, and further predicted and analyzed the HLA class II molecular binding peptides in the RBD sequence as predicted CD4⁺T cell epitopes, as shown in FIG. 2.

The inventors further predicted and analyzed the CD8⁺T cell epitopes in the RBD sequence, as shown in FIG. 3.

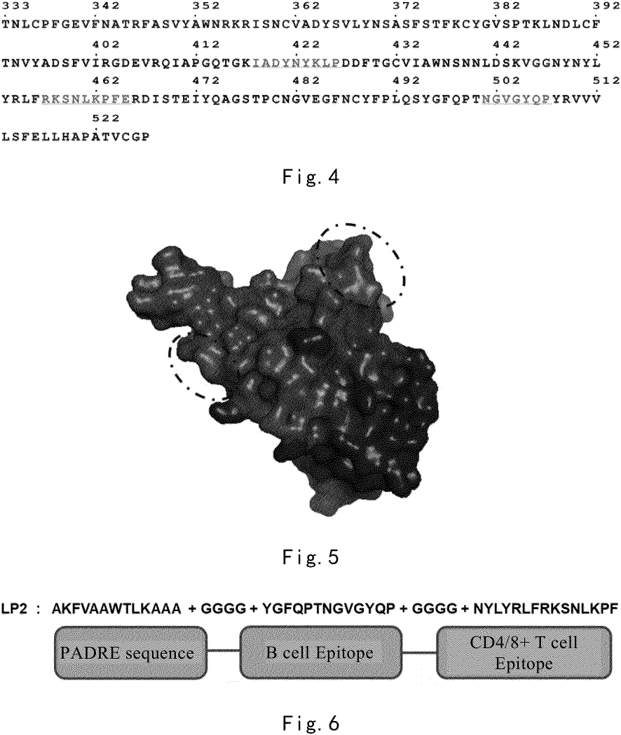

The invention also predicted the conformational and linear B cell epitopes in the RBD sequence using Discotope and BepiPred software, as shown in FIG. 4 and FIG. 5.

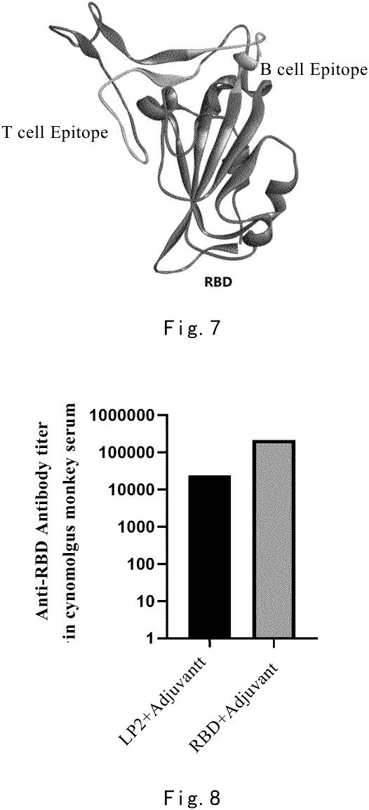

Based on the above information and analysis, the inventors finally designed the polypeptide LP2. The universal Th epitope PADRE sequence was introduced into LP2 and the B cell epitope and T cell epitope of the RBD region were connected in tandem through four glycines (GGGG). The T/B cell epitope was screened from the predicted T/B cell epitope regions according to their physical and chemical properties. The structural composition of LP2 is shown in FIG. 6, and the relative spatial positions of the B cell epitope and T cell epitope contained therein in the SARS-CoV-2 S protein RBD region are shown in FIG. 7. Its amino acid sequence is as follows:

LP2:

AKFVAAWTLKAAAGGGGYGFQPTNGVGYQPGGGGNYLYRLFRKSNLKPF
(49amino acids) (SEQ ID No: 1)

LP2 contains a CD8⁺T cell epitope, suggesting that it has better potential to stimulate antiviral response of killer T cells. LP2 contains a CD4⁺T cell epitope that binds with high affinity to 24 major HLA II molecular types in the population, suggesting that it can be presented by major HLA II molecules in the population. In order to further overcome the influence of MHC restriction on epitopes, the universal Th epitope PADRE sequence is introduced, ensuring a high population response rate and sufficient immunogenicity to stimulate a wide range of CD4⁺T cell effects and promote production of antiviral neutralizing antibodies by B cells. LP2 contains linear and conformational B cell epitopes, suggesting effective activation of B cell immune responses.

In addition, the PADRE sequence (AKFVAAWTLKAAA) used in LP2 was compared with the amino acid sequences of human proteins by Blastp software, and the E-Threshold was set to 10, and no similar sequence was found. This suggests that the introduced PADRE sequence has a lower risk of inducing antibodies against endogenous proteins in vivo after immunization.

Therefore, in terms of vaccine design, the tandem epitope polypeptide for SARS-CoV-2 helps to overcome the problems with traditional polypeptide vaccines of low immunogenicity and low response rate of the population, and takes into account the advantages of high specificity, high safety and easy rapid production and synthesis of traditional polypeptide vaccines.

### Example 2 Preparation of Polypeptide

In this example, the polypeptide LP2 was prepared by using a fully automatic solid-phase polypeptide synthesizer.

### Example 3 Immune Effect of LP2 Polypeptide Vaccine

In this example, cynomolgus monkeys were vaccinated with the polypeptide LP2 prepared in Example 2, and the immune effect of LP2 was evaluated.

Polypeptide LP2 and adjuvants (such as TiterMax) were mixed to prepare immune preparations, and injected subcutaneously in cynomolgus monkeys at multiple points for immunization. 14 days after the second immunization, antibody titer was measured by Bridging-ELISA method, and the ability of antiserum to block the binding of RBD and ACE2 was measured.

The neutralizing antibody was detected by competitive ELISA method, and the specific determination method is as follows: an ELISA plate was coated overnight with 10 µg/mL ACE2, and then blocked for use. The anti-peptide serum was diluted in different degrees with sample dilution buffer (1:128, 1:64, 1:32, 1:16, 1:8, and 1:4), then the diluted anti-serum in different degrees were incubated with Bio-RBD of 12 µg/mL at 37° C. for 1 hour. Then 100 µL of the reaction mixture was added into the wells of the blocked ACE2-coated ELISA plate, and incubated at 37° C. for 1 hour. Then the plate was washed, and HRP-Streptavidin A diluted in 1:10000 was added and incubated at 37° C. for 1 hour. After washing the plate, TMB was added for color development, and the plate was read at 450 nm wavelength after termination.

### Results

As shown in FIG. 8, after using LP2 polypeptide vaccine to immunize animals, a higher titer of antibodies against RBD was produced, indicating that LP2 has good immunogenicity and can better initiate immune response of body.

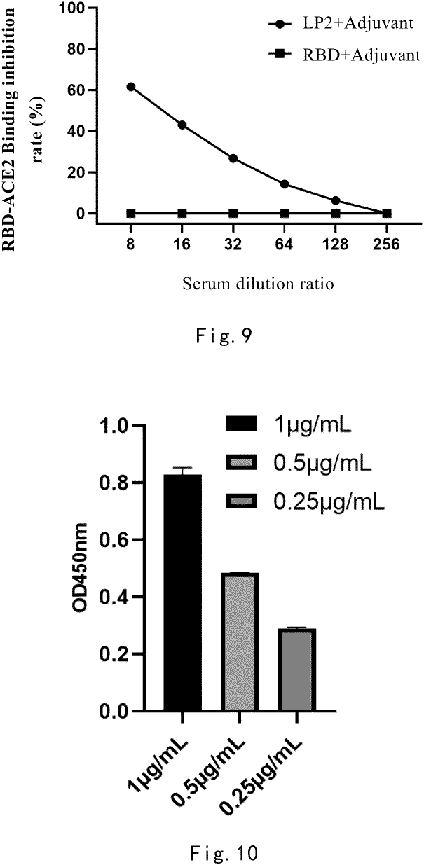

In addition, the antiserum produced by the LP2 polypeptide vaccine has the ability to block the binding of RBD to ACE2, that is, it has the effect of blocking the infection of the SARS-CoV-2 virus (FIG. 9).

Unexpectedly, the antiserum prepared with LP2 polypeptide vaccine can be observed a significant blocking effect on the binding of RBD to ACE2 at 8-64 times of dilution (for example, the inhibition rate at 8 times of dilution is slightly more than 60%, and the inhibition rate at 16 times of dilution is more than 40%).

In contrast, the antiserum produced by RBD immunization was not observed to block the binding of RBD to ACE2 at 8-64 times of dilution. A reasonable explanation is that RBD immunized cynomolgus monkeys did produce neutralizing antibodies, but produced more non-neutralizing antibodies that can bind RBD. Such antibodies instead promote RBD to form grid structure and bind to ACE2 in ELISA detection assay in vitro. It suggests that RBD without epitope specificity will produce more non-neutralizing antibodies when used for immunization, instead there is a risk of promoting SARS-CoV-2 invasion, while LP2 with high epitope specificity mainly produces neutralizing antibodies, which can more effectively block SARS-CoV-2 invasion and is significantly better than RBD immunization program.

### Example 4 In Vitro Binding Experiment of LP2 and ACE2

In this example, LP2 was labeled with biotin, and its binding ability with human ACE2 was detected by ELISA.

The specific determination method is as follows: the ELISA plate was coated overnight with 10ug/mL ACE2 and blocked, then biotin-labeled polypeptides of different concentrations (1, 0.5 and 0.25 µg/mL) were added. After incubated at 37° C. for 1.5 hours, HRP-Streptavidin A diluted in 1:5000 was added, and incubated at 37° C. for 1 hour. The plate was washed, then TMB was added for color development, and the plate was read at 450 nm wavelength after termination.

The results show that the polypeptide LP2 has a strong binding ability with ACE2 (FIG. 10), suggesting that the polypeptide LP2 itself has a potential blocking effect.

### Discussion

Polypeptide vaccine is one or more antigenic epitope fragments selected from highly immunogenic proteins for immunization. Due to the short amino acid chain of polypeptide vaccine, and thanks to the maturity of peptide synthesis technology, it facilitates rapid and large-scale in vitro synthesis and purification, and it is easy to ensure the purity and repeatability of each batch of products.

Meanwhile, computer-assisted vaccine design can quickly respond to emergent public health problems caused by novel viruses, and predict and screen suitable candidate peptides for vaccine development.

In addition, compared with other types of vaccines, polypeptide vaccines have clear epitopes, good stability, high purity and better safety. However, as the amino acid chain length is short, usually about 10-30 amino acids, polypeptide vaccines also face the problem of low immunogenicity, and often need to be modified or supplemented with adjuvants to produce a better immune response.

The design of polypeptide vaccines requires the help of computer-assisted vaccine design technology, which often requires comprehensive analysis of T/B cell epitopes, and structure and modification information of the target protein. Wherein, CD8+T cell epitopes can activate killer T cells to exert antiviral effects, and CD4+T cell epitopes mainly activate helper T cells to activate B cells to produce antiviral antibodies. Linear and conformational B cell epitopes can directly activate B cells to produce antibodies.

SARS-CoV-2 invades host cells by binding to human Angiotensin-convertion enzyme 2 (ACE2) protein through its surface Spike glycoprotein (S protein). Therefore, S protein is the preferred target protein for COVID-19 vaccine design, in the hope of inducing the production of neutralizing antibodies that can block virus invasion in the body. However, currently known vaccines are difficult to effectively trigger the body to produce a protective immune response against the coronavirus SARS-CoV-2.

Through research, the inventors unexpectedly discovered that based on the screened and sequence optimized antigenic polypeptide of the S protein of the coronavirus SARS-CoV-2, the vaccine polypeptide (such as LP2) with tandem B cell epitope, T cell epitope and PADRE sequence was designed, which can effectively induce the production of antiviral antibodies targeting and having a blocking effect on the RBD region of S protein in organism including primates. In addition, the vaccine polypeptide (such as LP2) of the present invention has an optimized structure and still retains a better binding effect with human ACE2, suggesting that it has the potential to directly block the interaction of S protein and ACE2.

Taking LP2 as an example, the antigenic polypeptide of the present invention is extended to 49 amino acids by epitope tandem, thereby enhancing the immunogenicity of the polypeptide. At the same time, by connecting CD4⁺T/CD8⁺T cell epitope and B cell epitope in S protein in tandem, it can better induce the body to product antiviral antibodies against and having a blocking effect on the RBD region of S protein and stimulate the antiviral cellular immunity of killer T cells. In addition, the PADRE sequence is introduced into LP2, which has high affinity with most HLA class II molecules, and can make the vast majority of the population respond and improve the immunogenicity and vaccination response rate of the vaccine.

The tandem epitope polypeptide vaccine of the present invention overcomes the problems of low immunogenicity and low population response rate with traditional peptide vaccines, while retaining its outstanding advantages of good safety, high epitope specificity and easy synthesis, and shows the effect and prospect of being superior to ordinary short peptide immunization schemes and direct immunization of RBD.

Therefore, the vaccine polypeptide of the present invention can be used to develop novel coronavirus polypeptide vaccines. It triggers cellular and humoral immunity in the human body to prevent and treat coronavirus SARS-CoV-2 infections and related diseases, including Corona virus disease 2019 (COVID-19).

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A vaccine polypeptide of novel coronavirus, which comprises the following elements in tandem: a universal Th epitope sequence, a B cell epitope sequence and a T cell epitope sequence, wherein the B cell epitope and the T cell epitope have amino acid sequences derived from the RBM region of the SARS-CoV-2 S protein.

2. The vaccine polypeptide of claim 1, wherein the vaccine polypeptide has a structure of Formula I or an oligomer comprising the structure of Formula I: 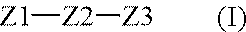
wherein,
Z1, Z2, and Z3 are each independently the universal Th epitope sequence, the B-cell epitope sequence, the T-cell epitope, or a combination thereof;
“-” is a chemical bond or linker;
and at least one of Z1, Z2, and Z3 is the universal Th epitope sequence; at least one is the B-cell epitope sequence; and at least one is the T-cell epitope.

3. The vaccine polypeptide of claim 1, wherein the universal Th epitope sequence comprises a PADRE sequence.

4. The vaccine polypeptide of claim 1, wherein the B cell epitope and/or the T cell epitope in the antigenic polypeptide has an amino acid sequence derived from the RBD region of the novel coronavirus S protein.

5. The vaccine polypeptide of claim 1, wherein in Formula I, Z1 is AKFVAAWTLKAAA (positions 1-13 in SEQ ID NO: 1); Z2 is YGFQPTNGVGYQP (positions 18-30 in SEQ ID NO: 1); Z3 is NYLYRLFRKSNLKPF (positions 18-30 in SEQ ID NO: 1).

6. The vaccine polypeptide of claim 1, wherein the RBM region refers to amino acids of positions 438-506 of the novel coronavirus RBD protein.

7. The vaccine polypeptide of claim 1, wherein the antigenic polypeptide is selected from the group consisting of:
(a) a polypeptide having the amino acid sequence shown in SEQ ID NO: 1;
(b) a derivative polypeptide formed by one or more amino acids addition, one or more amino acids substitution, or 1-3 amino acids deletion to the linking peptide of the amino acid sequence of the polypeptide in (a), and the derivative polypeptide has the same or substantially the same function as the polypeptide shown in SEQ ID NO: 1 before derivatization.
(c) a derivative polypeptide formed by one or more amino acids addition, one or more amino acids substitution, or 1-3 amino acids deletion to Z1, Z2 and/or Z3 of the amino acid sequence of the polypeptide in (a), and the derivative polypeptide has the same or substantially the same function as the polypeptide shown in SEQ ID NO: 1 before derivatization.

8. The vaccine polypeptide of claim 1, wherein the vaccine polypeptide can stimulate primates and rodents to produce neutralizing antibodies that block the binding of RBD to ACE2.

9. An isolated peptide set, wherein the peptide set comprises at least two vaccine polypeptides of novel coronavirus of claim 1.

10. A pharmaceutical composition, which comprises the vaccine polypeptide of novel coronavirus of claim 1 or a peptide set comprising at least two vaccine polypeptides of novel coronavirus, and a pharmaceutically acceptable carrier.

11. A method for the prevention of coronavirus SARS-CoV-2 infection or a related disease thereof, comprising administering the vaccine polypeptide of novel coronavirus of claim 1, or a peptide set comprising at least two vaccine polypeptides of novel coronavirus to a subject in need thereof.

12. The method of claim 11, wherein the coronavirus SARS-CoV-2 related disease is selected from the group consisting of respiratory infections, pneumonia and a complication thereof, and a combination thereof.

13. A cell preparation comprising: (a) immune cells immuno-activated with the vaccine polypeptide of novel coronavirus of claim 1; and (b) a pharmaceutically acceptable carrier.

14. The preparation of claim 13, wherein the immune cell is selected from the group consisting of dendritic cells, natural killer cells (NK), lymphocytes, monocytes/macrophages, granulocytes, and a combination thereof.

15. A method for generating an immune response against the coronavirus SARS-CoV-2, which comprises the steps of: administering the vaccine polypeptide of novel coronavirus of claim 1, a peptide set comprising at least two vaccine polypeptides of novel coronavirus to a subject in need thereof.


SEQUENCE LISTING (2 sequences)

SEQ ID NO: 1 - protein, 49 aa, Artificial sequence
  synthetic peptide
AKFVAAWTLKAAAGGGGYGFQPTNGVGYQPGGGGNYLYRLFRKSNLKPF

SEQ ID NO: 2 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT